(19) United States
(12) Patent Application Publication
(10) Pub. No.: US 2022/0111234 A1
(43) Pub. Date: Apr. 14, 2022

(54) PERSONAL AIR PURIFIER

(71) Applicant: Global Rise Enterprises Limited, Hong Kong (CN)

(72) Inventors: Lok Hang KEUNG, Hong Kong (CN); King Ho SO, Hong Kong (CN); Juanfang CAI, Hong Kong (CN); Chun Kit LIU, Hong Kong (CN)

(21) Appl. No.: 17/498,858

(22) Filed: Oct. 12, 2021

Related U.S. Application Data
(60) Provisional application No. 63/090,398, filed on Oct. 12, 2020.

(51) Int. Cl. A62B 23/02; A61L 9/22; B01D 24/00; B01D 39/04; B01J 21/06

(52) U.S. Cl. CPC A62B 23/02; A61L 9/22; A61L 2209/14; B01D 39/04; B01J 21/063; B01D 24/00

(57) ABSTRACT

A personal air-purifying (PAP) mask includes a fan assembly; a plasma driven catalyst (PDC) assembly including an anti-microbial catalyst, wherein the anti-microbial catalyst is incorporated within a nanofiber layer of the PDC assembly; and a pre-filter, wherein the pre-filter also includes the anti-microbial catalyst incorporated therewith; wherein the PDC assembly is positioned between the fan assembly and the pre-filter, and wherein the fan assembly, the PDC assembly, and the pre-filter are within a main body of the PAP mask.

### CROSS-REFERENCE TO RELATED APPLICATIONS

This application claims the benefit of U.S. Provisional Application No. 63/090,398, filed on Oct. 12, 2020, which is incorporated herein by reference.

### FIELD OF THE INVENTION

Embodiments of the present invention relate to a personal air purifier. Embodiments of the present invention relate to a personal air-purifying (PAP) mask integrated with a plasma driven catalyst (PDC) technology and further having an anti-microbial catalyst and a nanofiber pre-filter.

### BACKGROUND OF THE INVENTION

Disposable face masks and respirators are well known in the art. In the medical field, such masks are used in preventing contamination of a patient by the exhaled breath of healthcare personnel. However, the present public health pandemic has health care professionals seeking enhancement with respect to isolation precautions for routine care.

A powered air-purifying respirator (PAPR) is a type of respirator used to safeguard workers against contaminated air. It generally includes a battery-powered blower that provides positive airflow through a filter, cartridge, or canister to a hood or face piece. The type and amount of airborne contaminant will dictate the type of filter, cartridge, or canister required for the PAPR. Generally, traditional PAPR's are designed for higher level of protection and workers with specific needs of health care such as aggregates, casting, demolition, construction, facility sanitation, food safety, and industrial maintenance.

Conventional PAPR's may lead to difficulty in communicating due to their bulkiness and noises they generate. Conventional PAPR's may also be relatively heavy. Moreover, conventional PAPR's tend to not provide complete virus inactivation as their filter can generally only protect users from certain gases, particulates, or vapors. Furthermore, conventional PAPR's may require relatively high maintenance and cost.

An existing TiO₂-based photocatalytic coating is described in U.S. Pat. No. 8,529,831. The TiO₂-based coating of the '831 patent includes titanium dioxide and optionally includes one or more metals selected from Ti, Zn, Cu, La, Mo, W, V, Se, Be, Ba, Ce, Sn, Fe, Mg, and Al, and/or alloys, and/or oxides thereof. The photocatalytic property of the TiO₂-based coating is activated by irradiation from a UVA light tube with an intensity of 500 μW/cm². This UVA light source can be a UV light bulb, UV LED, or any source which can emit UV irradiation with wavelength from 320 nm to 400 nm, more preferably at 365 nm. The TiO₂-based coating activates the second oxidation of gaseous pollutant in the presence of a sufficient ozone supply and UV irradiation. Another function of the coating is to eliminate the excess ozone because such coating also has ozone-decomposing activity. In-situ elimination of excess ozone can avoid the leakage of these reactive molecules together with the purified gases. After passing through a second filter, the purified gases are ready to be exhausted back to the same indoor environment from where the polluted gases are collected or to another environment such as another enclosed environment or the atmosphere via an exhaust. The '831 patent discloses the pore size distribution of mesoporous TiO₂ thin film. The pore size of TiO₂ thin film is around 4 nm. The '831 patent discloses that TiO₂ thin films composed of small particles with the pore size of around 4-5 nm.

In certain existing photocatalytic oxidation systems, antimicrobial and air purification functions of the TiO₂ layer must be activated by ozone and UV-A. A relatively high level of ozone may be released by existing photocatalytic systems as a by-product of the photocatalytic oxidation. While ozone itself may be useful in neutralizing volatile organic compounds (VOC's) and as an anti-microbial agent, relatively high levels of ozone can be undesirable for personal air purifiers as these relatively high levels of ozone may be toxic to the wearer.

A plasma driven catalyst reactor is disclosed in U.S. Pat. No. 9,138,504. The plasma driven catalyst reactor disinfects, cleans, and purifies air to remove the air pollutants and improve indoor air quality. The reactor of the '504 patent generally includes a pre-filter, an electric fan, and a plasma reactor with catalyst inside. The plasma technology used in the '504 patent is based on dielectric barrier discharge (DBD) plasma. The non-equilibrium discharge can be handily operated at atmospheric pressure conditions. DBD is formed between two parallel electrodes separated by an insulating dielectric barrier. The most important characteristic of barrier discharges is the non-equilibrium plasma conditions which is much simpler compared with other alternative plasma technologies like electron beam, low pressure discharges, and pulsed high pressure corona discharges. The DBD plasma process uses a high voltage alternating current (AC) ranging from 4 kV to 30 kV with the frequency ranging from several hundred hertz (Hz) to few hundred kilo hertz (kHz). This sufficiently high voltage is used to ionize the media in the gap between the two electrodes, which contains a number of components like electrons of different energy, positive and negative ions, and neutral particles. These ionized components can deeply degrade the VOC's and other air pollutants into non-harmful products like CO₂ and H₂O.

However, DBD plasma generates ozone and other toxic by-products during the disinfection and purification process. A catalyst is deposited on the plasma reactor to remove those toxic by-products. The catalyst used in the '504 patent is titanium dioxide (TiO₂) based catalyst. This TiO₂-based coating has a plurality of mesoporous structures with a pore size of 2-20 nm so the total effective surface area is greatly increased. The TiO₂ catalyst may be doped with other elements, such as Ti, Zn, Cu, Mn, La, Mo, W, V, Se, Ba, Ce, Sn, Fe, Mg, Au, Pt, Co, Ni or Pd, or its oxides, or its alloys to enhance its photocatalytic performance. This TiO₂ based catalyst can be activated in the plasma reactor without additional UV light irradiation. The generated ozone and other byproducts from the DBD plasma can be eliminated by the TiO₂ based catalyst. The position of the catalyst can be located on the surface of the electrodes, between electrodes, or at the back end of the plasma reactor.

In the '504 patent, a plasma reactor for purifying air comprises: at least two spaced plasma electrodes for generating plasma within a plasma zone between the at least two spaced plasma electrodes by an alternating current voltage; at least one insulating dielectric layer; at least one photocatalyst layer; and at least one air inlet and at least one air outlet for allowing air passing through the plasma; wherein the insulating dielectric layer is formed on at least one of the spaced plasma electrodes; wherein the photocatalyst layer is deposited on the insulating dielectric layer; and wherein the photocatalyst layer is in face of the plasma. The photocatalyst layer is located within the plasma zone between the at least two spaced plasma electrodes. The photocatalyst layer is located at the air inlet of the plasma reactor, or the air outlet of the plasma reactor. At least one surface of the photocatalyst layer is exposed to the plasma zone and in contact with the plasma. The plasma reactor may comprise a casing; a filter; and an orientation air deflector.

There remains a need in the art for a personal air purifier with improved air purifying and anti-microbial properties.

### SUMMARY OF THE INVENTION

An embodiment of the present invention provides a personal air-purifying (PAP) mask including a fan assembly; a plasma driven catalyst (PDC) assembly including an anti-microbial catalyst, wherein the anti-microbial catalyst is incorporated within a nanofiber layer of the PDC assembly; and a pre-filter, wherein the pre-filter also includes the anti-microbial catalyst incorporated therewith; wherein the PDC assembly is positioned between the fan assembly and the pre-filter, and wherein the fan assembly, the PDC assembly, and the pre-filter are within a main body of the PAP mask.

According to one or more embodiments of the present invention, in an in-use configuration, the fan assembly pulls ambient air into the mask body, with the air first passing through the pre-filter before passing through the PDC assembly before reaching a wearer of the PAP mask. In one or more embodiments, the PAP mask of the present invention further comprises an exhaust, where the exhaust may include a filter for filtering the air being exhaled by the wearer. In yet other embodiments, the PAP mask of the present invention further comprises a divider that separates the PDC assembly, the fan assembly, and the pre-filter from electronic components.

### BRIEF DESCRIPTION OF THE DRAWINGS

Advantages of the present invention will become better understood with regard to the following description, appended claims, and accompanying drawings wherein:

FIG. 1 is a schematic of a personal air-purifying (PAP) mask integrated with a plasma driven catalyst (PDC) technology having an anti-microbial catalyst incorporated in a layer of nanofiber and in a nanofiber pre-filter, according to one or more embodiments of the invention;

FIG. 2 is a schematic of an alternative personal air-purifying (PAP) mask integrated with a plasma driven catalyst (PDC) technology having an anti-microbial catalyst incorporated in a layer of nanofiber and in a nanofiber pre-filter, according to one or more embodiments of the invention;

FIG. 3 is a schematic of a PDC-based disinfecting and purifying apparatus, according to one or more embodiments of the invention;

FIG. 4 is a schematic of a PDC-based plasma reactor with catalyst layers coated on two electrodes, according to one or more embodiments of the invention;

FIG. 5 is a schematic of a PDC-based plasma reactor with a catalyst layer located between two electrodes, according to one or more embodiments of the invention;

FIG. 6 is a schematic of a PDC-based plasma reactor with a catalyst layer located at a back end of the plasma reactor, according to one or more embodiments of the invention; and

FIG. 7 is a graph of the removal efficiency of a plasma driven catalyst (PDC) reactor according to one or more embodiments of the invention, without the nanofiber pre-filter.

### DETAILED DESCRIPTION OF THE INVENTION

Embodiments of the present invention relate to a personal air-purifying (PAP) mask. The personal air-purifying mask includes an integrated plasma driven catalyst (PDC) assembly that includes an anti-microbial catalyst. The anti-microbial catalyst is effective against bacteria and virus and may be referred to herein as an anti-bacteria/anti-virus catalyst. In an embodiment, the anti-microbial catalyst includes, but is not limited to, a TiO₂-based catalyst, and may also be referred to as a photocatalyst (PCO). The anti-microbial catalyst of the PDC assembly may be incorporated within a nanofiber layer. The personal air-purifying mask further includes a pre-filter. The pre-filter may be a nanofiber pre-filter. The pre-filter may also include an anti-microbial catalyst (e.g. a TiO₂-based catalyst).

It has advantageously been found that the personal air-purifying mask of embodiments of the present invention provides a complete, compact, lightweight, and user-friendly solution for protection against particulates, VOC's, bacteria, and viruses. Moreover, because the personal air-purifying mask of embodiments of the present invention is lightweight and reusable, the personal air-purifying mask can be used in general activities. Also, the personal air-purifying mask of embodiments of the present invention satisfies air purification performance tests, such as the One Pass Filtration test with over 99% filter efficiency. The personal air-purifying mask of embodiments of the present invention is highly suitable for use as a personal air purifier, while also providing antibacterial and antiviral properties.

As will be further described below relative to certain embodiments, one or more embodiments of the present invention provide a personal air-purifying mask that includes a plasma driven catalyst (PDC) assembly, a fan assembly, a pre-filter, and a mask housing.

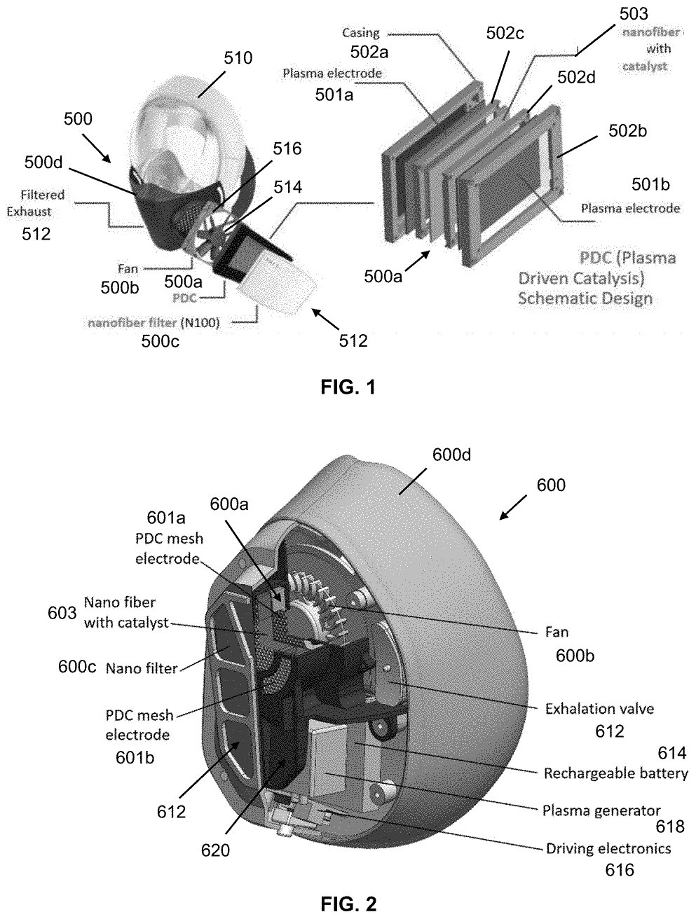

With reference to FIG. 1, a personal air-purifying mask 500 includes a plasma driven catalyst (PDC) assembly 500a, a fan assembly 500b, a pre-filter 500c, and a mask housing 500d. Though FIG. 1 shows PDC assembly 500a, fan assembly 500b, and pre-filter 500c outside of mask housing 500d, it should be appreciated that this is for representative purposes only. PDC assembly 500a, fan assembly 500b, and pre-filter 500c will be within mask housing 500d in the in-use configuration.

As shown in FIG. 1, the PDC assembly 500a, which may also be referred to herein as a PDC component 500a or a plasma reactor interchangeably, the fan assembly 500b, which may also be referred to as a fan module 500b, and the pre-filter 500c, which may also be referred to as a nanofiber pre-filter 500c, are positioned in layers. As discussed above, in the in-use configuration, these layers would be secured in place, which may also be referred to as being held together, by the mask housing 500d.

The PDC component 500a is sandwiched between the pre-filter 500c and the fan module 500b. The fan module 500b is located on the inner side, that is, closet to the wearer 510. The pre-filter 500c is located on the external side, that is, distal to the wearer 510. Based on this configuration, the atmospheric non-filtered air, shown with arrow 512, first enters through the pre-filter 500c, and then the PDC component 500a, before reaching the interior of the personal air-purifying mask 500.

With further description of PDC component 500a, as shown in FIG. 1, plasma driven catalyst (PDC) component 500a includes a variety of components positioned in layers. A middle layer 503, which can be a nanofiber layer 503, is located between two spaced plasma electrodes 501a, 501b. Where the middle layer 503 is a nanofiber layer 503, nanofiber layer 503 may be made from any nanofibers suitable for the filtering function. In one or more embodiments, the nanofibers may be made of polyacrylonitrile (PAN). PAN-DMF can be used as a raw material to synthesize the nanofibers.

The middle layer 503 should provide a relatively large surface area per unit weight. The nanofiber layer may have a thickness of 0.3 mm, or approximate thereto, and may be composed of nanofibers with diameter ranges from 200 nm to 700 nm. This relatively large surface area per unit weight of the middle layer 503 allows middle layer 503 to serve as an effective carrier for antimicrobial catalyst incorporated therewith. The antimicrobial catalyst is effectively supported by the middle layer 503. It has advantageously been found that incorporating the anti-microbial catalyst into a layer of nanofibers enhances the anti-microbial activity and reduces production of harmful by-products (e.g. ozone) without activation by external sources, such as UV.

As mentioned above, the middle layer 503 of the PDC assembly 500a includes an anti-microbial catalyst incorporated therewith. An exemplary anti-microbial catalyst is a titanium dioxide (TiO₂)-based catalyst. The TiO₂-based catalyst may be doped with one or more other elements, such as Ti, Cu, Ce, Co, Ni, and/or alloys thereof, and/or oxides thereof, and/or combinations thereof. The anti-microbial efficiency of the PDC assembly 500a and the overall mask 500 can be enhanced by optimizing the mesophorous structure of the TiO₂-based catalyst through incorporating the catalyst in a layer of nanofibers. The mesophorous structure of the nanofiber layer may provide an average pore size of from 2 nm to 20 nm to increase the total surface area of the catalyst. In other embodiments, the nanofiber layer of the present invention provides an average pore size of from 4 nm to 5 nm. In another embodiment, the average pore size of the nanofiber layer is 4 nm. The nanofiber layer that is incorporated with catalyst is able to oxidize about 82% to about 84% of gaseous pollutants into harmless gases within 5 mins. Suitable mesophorous structures are disclosed in U.S. Pat. No. 8,529,831, and the disclosure thereof is incorporated herein by reference in its entirety. Other details of the TiO₂-based photocatalytic catalyst and/or coating thereof may be described in U.S. Pat. No. 8,529,831. In one embodiment, the anti-microbial catalyst is a photocatalyst. In accordance with embodiments of the present invention, an external source, e.g. UV and ozone, is not required for activation of the photocatalyst.

As further description of the various components of PDC component 500a, respective casing or housing units may be utilized between and/or outside of each of the various layers. More specifically, a first casing or housing unit 502a can support the first plasma electrode 501a and a second casing or housing unit 502b can support the second plasma electrode 501b. A third casing or housing unit 502c may be positioned between the first plasma electrode 501a and the middle layer 503. A fourth casing or housing unit 502d may be positioned between the second plasma electrode 501b and the middle layer 503. All of the various casing or housing units can be secured together with one or more suitable fasteners (not shown). The first casing or housing unit 502a and the second casing or housing unit 502b fully encompass all components of PDC component 500a.

For operation of the PDC component 500a, a power supply (not shown), such as an AC power supply, which can be a rechargeable battery, is connected to a plasma generator in order to provide a voltage alternating current to the spaced plasma electrodes 501a, 501b. Suitable voltages ranges from 1 kV to 5 kV. This generates a plasma within a plasma zone located between the spaced plasma electrodes 501a, 501b. The middle layer 503 with the anti-microbial catalyst is in contact with the plasma. When polluted air from the air inlet passes through the plasma, the polluted air is purified and disinfected, and the purified air is released into the mask housing 500d.

With further description of fan module 500b, fan module 500b is electronic and may be powered by a battery (not seen), which may be a rechargeable battery. Fan module 500b serves to pull air into the mask housing 500d, which may also be referred to as introducing airflow or providing positive airflow into the mask housing 500d. Fan module 500b may include a fan portion 514 that is positioned within an outer housing portion 516. The inner diameter of the outer housing portion 516 may be similar to the outer diameter of the fan portion 514, while including suitable space for travel of the fan portion 514. The outer housing portion 516 generally serves to protect the blades of the fan portion 514 from contacting other components.

With further description of pre-filter 500c, pre-filter 500c is a multi-functional filter that provides low air pressure drop, high dust holding capacity, high VOC's removal, and bacteria killing ability. Pre-filter 500c may be made from a variety of materials, including suitable nanofibers as readily known by a skilled person in the art. In one embodiment, the nanofibers are made of polyacrylonitrile (PAN). PAN-DMF may be used as a raw material to synthesize the nanofibers. Pre-filter 500c may have an N100 or ASTM F2100-2020 classification.

The material for the pre-filter 500c (e.g. nanofibers) may include the anti-bacteria/anti-virus catalyst (e.g. a TiO₂-based catalyst) incorporated therein or therewith. This may include manufacturing the pre-filter 500c with the TiO₂-based catalyst incorporated therewith. With this incorporation, particularly during manufacture, concerns over the wearer inhaling the TiO₂-based catalyst can be resolved. In other embodiments, the TiO₂-based catalyst might be incorporated with the pre-filter 500c after the pre-filter 500c is manufactured.

As further shown in FIG. 1, the mask housing 500d, which may also be referred to as the main body 500d, is of a suitable size and shape as to be placed over the nose and mouth (not seen) of the wearer 510. Mask housing 500d may be coupled with a suitable strap or straps or other suitable component for maintaining the position of mask housing 500d relative to the nose and mouth of the wearer 510.

Mask housing 500d further includes an exhaust 512. The exhaust 512 of the mask housing 500d may include a suitable filter component (not seen) so as to filter the air being exhaled by the wearer 510 before the air reaches the atmospheric environment. Suitable filter components for this purpose will be generally known to the skilled person.

With reference to FIG. 2, a personal air-purifying mask 600 includes a plasma driven catalyst (PDC) assembly 600a, a fan assembly 600b, a pre-filter 600c, and a mask housing 600d. PDC assembly 600a, fan assembly 600b, and pre-filter 600c are within mask housing 600d in the in-use configuration.

As shown in FIG. 2, the PDC assembly 600a, which may also be referred to as a PDC component 600a or a PDC reactor herein, the fan assembly 600b, which may also be referred to as a fan module 600b, and the pre-filter 600c, which may also be referred to as a nanofiber pre-filter 600c, are positioned in layers. In the in-use configuration, these layers are secured in place, which may also be referred to as being held together, by the mask housing 600d.

The PDC component 600a is sandwiched between the pre-filter 600c and the fan module 600b. The fan module 600b is located on the inner side, that is, closet to the wearer. The pre-filter 600c is located on the external side, that is, distal to the wearer. Based on this configuration, the atmospheric non-filtered air, shown with arrow 612, first enters through the pre-filter 600c, and then the PDC component 600a, before reaching the interior of the personal air-purifying mask 600.

With further description of PDC component 600a, as shown in FIG. 2, plasma driven catalyst (PDC) component 600a includes a variety of components positioned in layers. A middle layer 603, which can be a nanofiber layer 603, is located between two spaced plasma electrodes 601a, 601b. The middle layer 603 of the PDC assembly 600a includes an anti-microbial catalyst incorporated therewith.

The properties and details of PDC component 600a, middle layer 603, spaced plasma electrodes 601a, 601b, and anti-microbial catalyst generally correspond to the above disclosed PDC component 500a, middle layer 503, spaced plasma electrodes 501a, 501b, and anti-microbial catalyst. The above disclosure is therefore incorporated here as well relative to PDC component 600a, middle layer 603, spaced plasma electrodes 601a, 601b, and anti-microbial catalyst.

Similarly, the properties and details of the fan assembly 600b and the pre-filter 600c generally correspond to the above disclosed fan assembly 500b and pre-filter 500c. The above disclosure is therefore incorporated here as well relative to fan assembly 600b and the pre-filter 600c.

As further description of the mask housing 600d, the mask housing 600d, which may also be referred to as the main body 600d, is of a suitable size and shape as to be placed over the nose and mouth (not seen) of the wearer. Mask housing 600d may be coupled with a suitable strap or straps or other suitable component for maintaining the position of mask housing 600d relative to the nose and mouth of the wearer.

Mask housing 600d further includes an exhaust 612. The exhaust 612 of the mask housing 600d may include a suitable filter component (not seen) so as to filter the air being exhaled by the wearer 610 before the air reaches the atmospheric environment. Suitable filter components for this purpose will be generally known to the skilled person. As shown in FIG. 2, the exhaust 612 may be an exhalation valve 612. After the filtered air enters the internal portion of main body 600d to be breathed in by the wearer, the air exhaled by the wearer leaves exhaust 612 to return to the atmospheric environment.

For operation of the PDC component 600a and fan assembly 600b, a power supply 614, such as an AC power supply, which can be a rechargeable battery 614, provides a voltage alternating current to the spaced plasma electrodes 601a, 601b. This generates a plasma within a plasma zone located between the spaced plasma electrodes 601a, 601b. The middle layer 603 with the anti-bacteria/anti-virus catalyst is in contact with the plasma. When polluted air from the air inlet passes through the plasma, the polluted air is purified and disinfected, and the purified air is released into the mask housing 600d.

As will be generally understood by the skilled person, the PDC component 600a, the fan assembly 600b, and the power supply 614 may be electronically coupled with the driving electronics 616 that are suitable to operate the various components.

As shown in FIG. 2, mask housing 600d may include a plasma generator 618. The plasma generator 618 generally serves to convert DC voltage of a rechargeable battery to high voltage alternate current in ranges from 1 kV to 5 kV. The high voltage output is connected to the PDC mesh electrodes 601a, 601b, by which plasma is generated between the two PDC mesh electrodes 601a, 601b.

As shown in FIG. 2, mask housing 600d may include a divider 620 to separate certain components. The divider 620 may be utilized to separate the breathing components (e.g. PDC component 600a, fan assembly 600b, and pre-filter 600c) from the non-breathing or electronic components (e.g. power supply 614, driving electronics 616, and plasma generator 618). The divider 620 may include housing portions specifically adapted to hold PDC component 600a and fan assembly 600b. The divider 620 may include an opening corresponding to the air flow path from pre-filter 600c to PDC component 600a and then to fan assembly 600b.

As discussed above, the personal air-purifying mask (e.g. mask 500, mask 600) of the present disclosure can satisfy the one pass filtration test with over 99% filter efficiency. The personal air-purifying mask of the present disclosure also significantly reduces the production of harmful by-products, e.g. ozone, on the order of only producing <5 ppb of ozone compared to certain conventional apparatuses that produce ppm of ozone. The personal air-purifying mask of the present disclosure also provides improved efficiency for anti-microbial removal and the removal of VOC's.

The synergy effect of the herein described features of the present invention strengthen the function and expand the available uses as a personal air-purifying mask. Moreover, the personal air-purifying mask of the present disclosure can be utilized to substitute disposable face masks and conventional PAPR's.

As further description of the PDC component, other details of a PDC component may be described in U.S. Pat. No. 9,138,504, which is incorporated herein by reference in its entirety.

Though the embodiments of FIG. 1 and FIG. 2 do not show dielectric layers for the utilization of dielectric barrier discharge (DBD) plasma, other embodiments of the present invention may utilize DBD based PCD assembly.

A dielectric barrier discharge (DBD) plasma assembly generally comprises two parallel spaced electrodes, and one or two dielectric barriers. The electrode is made of electrically conductive materials which may be in form of rods, tubes, pipe, foils, films, plates, or mesh. The distance between the two electrodes ranges from a few millimeters to one hundred millimeters. The electrodes are separated by the dielectric barriers and these barriers are either attached to the electrodes or inserted between two electrodes. A high voltage alternating current from 1 kV to 4 kV with the frequency ranging from several hundred hertz (Hz) to a few hundred kilo hertz (kHz) with power <0.3 W is applied on the electrodes to generate the DBD plasma inside the reactor.

The DBD based PDC assembly is able to be operated in the ambient conditions, i.e. room temperature, atmospheric pressure, and atmospheric relative humidity. The removable gaseous pollutants include but are not limited to NO_{R}, SO₂, H₂S, formaldehyde, NH₃, volatile organic compounds (VOC's), organic odors, and airborne bacteria and viruses. The combination of plasma and catalyst incorporated into nanofiber layers of the present invention has a synergic effect on further enhancing disinfection and purification of air and also has low toxic by-products emission. Consequently, the plasma technology which combines plasma with catalyst, can minimize or even eliminate those drawbacks of certain existing plasma technology.

The combination of plasma and catalyst layer of the present invention for air treatment is associated with advantages, such as higher energy efficiencies, low power consumption, high mineralization rates, and absence of by-product formation. Plasma driven catalytic air cleaning technology exhibits highly efficient purification by decomposing a large range of toxic molecules, including but not limited to, formaldehyde, methanol, into CO₂ and H₂O at low temperature. Changing plasma characteristics can eventually result in enhancing the production of new active species and increasing the oxidizing power of the plasma discharge. Plasma discharges also affect catalyst properties such as a change in chemical composition, enhancement in surface area, or change of catalytic structure. The catalyst in the plasma zone is activated by the plasma and by other activation mechanisms, but necessary, include ozone, UV, local heating, changes in work function, activation of lattice oxygen, adsorption/desorption, creation of electron-hole pairs, and direct interaction of gas-phase radicals with adsorbed pollutants. Besides assisting to degrade the gas pollutants in the plasma reactor, the activated catalyst can also degrade the toxic by-products generated from the plasma. Thus, the plasma driven catalyst technology of the present invention has much higher air purification efficiency and lower toxic by-products emission than using plasma only, or other air purification technologies.

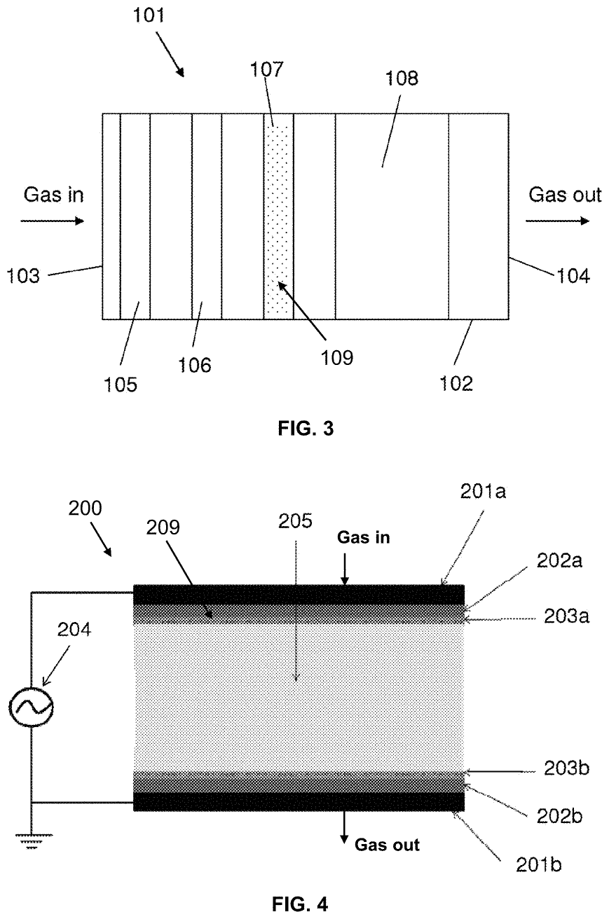

With reference to FIG. 3, a plasma driven catalyst disinfecting and purifying apparatus 101, which may also be referred to as PDC component 101, includes a casing 102 having an air inlet 103, and an air outlet 104, an electric fan 105, an orientation air deflector 106, a pre-filter 107, and a plasma reactor 108. The casing 102 encloses the electric fan 105, the orientation air deflector 106, the pre-filter 107, and the plasma reactor 108. The electric fan 105 generates airflow. The orientation air deflector 106 orientates the direction of the airflow. The pre-filter 107 removes air particulates. The plasma reactor 108 generates plasma for disinfecting and purifying air. The pre-filter 107 includes an anti-microbial catalyst 109 (e.g. a TiO₂-based catalyst 109) incorporated therewith.

With reference to FIG. 4, a PDC component 200 includes a pair of spaced plasma electrodes 201a and 201b, two insulating dielectric layers 202a, 202b, two photocatalyst layers, 203a, 203b, an AC power supply 204, an air inlet for gas in, and an air outlet for gas out. The gas enters the first plasma electrode 201a and exits the second plasma electrode 201b. The spaced plasma electrodes 201a, 201b are positioned in parallel with each other with a distance between. The insulting dielectric layer 202a is positioned on the spaced plasma electrode 201a and in face of the spaced plasma electrode 201b. Similarly, the insulting dielectric layer 202b is positioned on the spaced plasma electrode 201b and in face of the spaced plasma electrode 201a. The photocatalyst layer 203a may be coated on the insulting dielectric layer 202a, and the photocatalyst layer 203b may be coated on the insulting dielectric layer 202b. The photocatalyst layer 203a and the photocatalyst layer 203b may be a nanofiber 209 incorporated with photocatalyst, which are similar to the above-described nanofiber layer 503.

This is such that the photocatalyst layer 203a is in face of the spaced plasma electrode 201b while the photocatalyst layer 203b is in face of the spaced plasma electrode 201a. When the AC power supply 204 provides high voltage alternating current to the spaced plasma electrodes 201a and 201b, a plasma 205 is generated within a plasma zone located between the spaced plasma electrodes 201a and 201b. Both of the photocatalyst layers 203a and 203b are in contact with the plasma 205. When polluted air from the air inlet passes through the plasma 205 in the plasma reactor, the polluted air is purified and disinfected, and the purified air is released out from the air outlet. The photocatalyst layer 203b is a layer of nanofiber incorporated with anti-microbial catalyst (e.g. a photocatalyst or a TiO₂-based catalyst).

Where the photocatalyst layers are directly coated on the insulting dielectric layers, the photocatalyst layers can be effectively activated by the plasma in the plasma reactor without additional UV light irradiation to generate free radicals, which enable to decompose air pollutants such as VOC into non-harmful products like water and carbon dioxide, thereby further enhancing the air pollutant removal efficiency. Since the photocatalyst is in contact with the plasma, the efficiency of free radical generation is further increased under such reactive plasma environment. In addition, ozone or other harmful byproducts generated from the plasma are also eliminated by the free radicals.

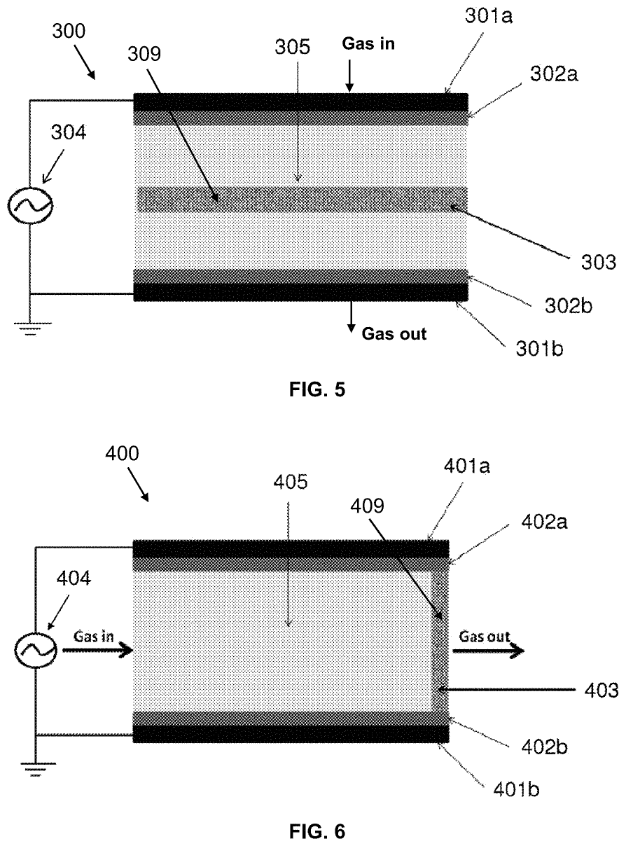

With reference to FIG. 5, a PDC component 300 includes a photocatalyst layer 303 is located in a plasma zone between a pair of plasma spaced electrodes, 301a and 301b, and placed in substantially parallel with the pair of plasma spaced electrodes, 301a and 301b. The gas enters the first plasma electrode 301a and exits the second plasma electrode 301b. The photocatalyst layer 303 is immersed and in contact with a plasma 305 generated by the pair of the plasma spaced electrodes such that the photocatalyst layer 303 is effectively activated by the plasma 305 to generate free radicals for decomposing air pollutants and eliminating ozone and other harmful by-products released from the plasma 305 without additional UV light irradiation. The photocatalyst layer 303 may be incorporated with nanofibers 309, which is similar to the above-described nanofiber layer 503. Insulating dielectric layers 302a and 302b are coated on the pair of plasma spaced electrodes, 301a and 301b respectively. An AC voltage is provided to the electrodes by an AC power supply 304 connected to the electrodes. The photocatalyst layer together with the nanofibers 309 may have a thickness ranging of from 10 μm to 500 μm. In one embodiment, the thickness of the photocatalyst 303 and the nanofibers 309 is 300 μm, or approximate thereto. The insulating dielectric layers 302a, 302b may have a thickness ranging of from 1 mm to 5 mm.

With reference to FIG. 6, a PDC component 400 includes a photocatalyst layer 403 located at the back end of the plasma reactor, and covering the air outlet of the plasma reactor. The surface of the photocatalyst layer 403 is exposed to a plasma zone between a pair of plasma spaced electrodes, 401a and 401b, and in contact with a plasma 405 such that the photocatalyst layer 403 is effectively activated by the plasma 405 to generate free radicals for decomposing air pollutants and eliminating ozone and other harmful by-products released from the plasma 405 without additional UV light irradiation. The photocatalyst 403 may be incorporated with nanofibers 409, which is similar to the above-described nanofiber layer 503. Insulating dielectric layers 402a and 402b are coated on the pair of plasma spaced electrodes, 401a and 401b respectively. An AC voltage is provided to the electrodes by an AC power supply 404 connected to the electrodes.

In any of the above embodiments, the sol-gel method may be used to coat the catalyst on the respective layer. The precursor of the photocatalyst with other chemicals may be formed and mixed well to form a pre-photocatalyst solution. Then the coating may be formed on the dielectric layer by dip coating. After that, the coating may be annealed in a furnace to form the photocatalyst layer.

As should be appreciated from the above disclosure, embodiments of the present invention relate to a personal air-purifying (PAP) mask utilizing plasma driven catalyst (PDC) technology integrated with an anti-bacteria/anti-virus catalyst and a nanofiber pre-filter. One or more embodiments of the personal air-purifying mask may have one or more of the following characteristics:

a) Improved precautions of routine care relative to air purification, antibacterial, and antiviral properties;

b) May include a plasma driven catalyst (PDC) component having a photocatalytic layer and a nanofiber pre-filter;

c) The PDC component may include two mesh plate electrodes in parallel and catalyst (“PCO” e.g. photocatalyst and TiO₂-based catalyst) incorporated in a nanofiber filter layer, with the PDC component sandwiched between the two electrodes;

d) The nanofiber filter with PCO may be fixed between electrodes in the component; the nanofibers may have diameters ranging from 200 nm to 700 nm;

e) The electronic fan module may continually supply positive air pressure to the main housing body to maintain positive pressure in the PAP system; the main housing body can provide personal respiratory protection by preventing ambient air from entering the user's mask; the volume flow rate of the air is or is higher than 72 L/min;

f) The nanofiber pre-filter material may be fabricated from nanofibers; the diameter of the nanofibers may range from 200 nm to 700 nm;

g) The filter efficiency of the pre-filter together with the PDC component achieve over 99.9%;

h) The personal air-purifying (PAP) mask may be able to kill no less than 99% of bacteria (e.g. E. coli and Staphylococcus Aureus); and

i) The personal air-purifying (PAP) mask may be able to kill no less than 99% of virus (e.g. H1N1).

### EXAMPLES

### Comparative Example

In order to demonstrate the advantage of the present disclosure, certain testing was done for a plasma driven catalyst (PDC) reactor according to the present invention but without the nanofiber pre-filter (i.e. the nanofiber pre-filter as described above relative to embodiments of the present disclosure).

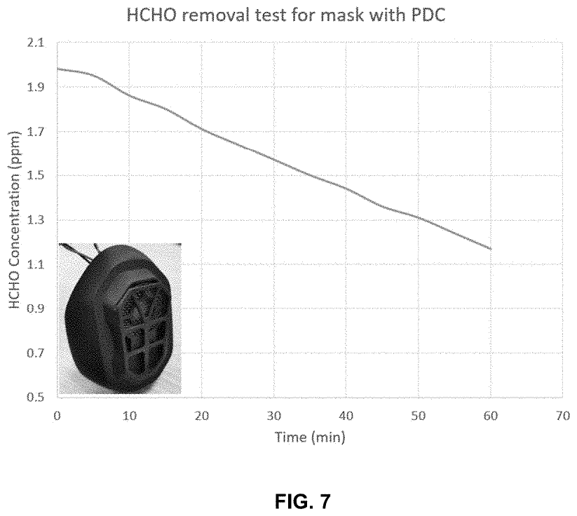

FIG. 7 shows a graph of the removal efficiency for formaldehyde (HCHO) for a plasma driven catalyst reactor without the nanofiber pre-filter.

### Example 1

The removal efficiency of the present personal air purifier having a plasma driven catalyst reactor with the nanofiber pre-filter was tested under the removal test of bacterial reference to technical standard for disinfection by 2002 Ministry of Health of the People's Republic of China. Suspension of test bacteria Staphylococcus Aureus (S. Aureus) and Escherichia Coli (E. Coli) were respectively incubated and prepared, filtered with sterile absorbent cotton and diluted with nutrient broth medium, to required concentrations. The bacteria suspension was sprayed using a cold atomizer into a 1 m³ test chamber that was controlled under a temperature of from 20° C. to 25° C. and a humidity from 50% to 70%. A fan of the present personal air purifier was switched on. Bacterial samples for performance tests were collected by a liquid impingement sampler in the chamber after operation of the present invention for 30 minutes, then impregnated medium solution was prepared and cultured in an incubator under 37° C. for 48 hours and the final results were observed. A control sample was prepared as in the above procedures without switching on the fan of the present invention; the bacterial sample was collected after 30 minutes.

Table 1 shows the result of the bacteria removal efficiency test for Staphylococcus Aureus (S. Aureus) and Escherichia Coli (E. Coli) of the plasma driven catalyst reactor of the present invention with the nanofiber filter. It is shown that the removal rate of S. Aureus is up to 2.3×10⁷ CFU/m³ for 30 min of time, or more than 6.388×10⁴ CFU/m³ for every 5 s.

TABLE 1

| | | | Present | |
|---|---|---|---|---|
| Bacterial | | | invention (fan | |
| sample | Unit | Control | switched on) | % Removal |
| S. Aureus | CFU/m³ | 4.2 × 10⁷ | 1.9 × 10⁷ | 55% |
| E. Coli | | 5.8 × 10⁷ | 3.3 × 10⁷ | 43% |

In light of the foregoing, it should be appreciated that the present invention significantly advances the art by providing an improved personal air purifier. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

What is claimed is:

1. A personal air-purifying (PAP) mask comprising
a fan assembly;
a plasma driven catalyst (PDC) assembly including an anti-microbial catalyst incorporated in a nanofiber layer,
a pre-filter also having an anti-microbial catalyst incorporated therein; and
a main body;
wherein the PDC assembly is positioned between the fan assembly and the pre-filter; and the fan assembly, the PDC assembly, and the pre-filter are housed within the main body of the PAP mask; and
wherein the fan assembly pulls air into the main body, with the air first passing through the pre-filter followed by passing through the PDC assembly before the air reaching a wearer of the PAP mask.

2. The PAP mask of claim 1, wherein the anti-microbial catalyst of the nanofiber layer and the anti-microbial catalyst of the pre-filter both comprise a titanium dioxide (TiO₂)-based catalyst.

3. The PAP mask of claim 2, wherein the TiO₂-based catalyst is doped with an additional element selected from Ti, Cu, Ce, Co, and Ni, an alloy of the additional element, an oxide of the additional element, or a combination thereof.

4. The PAP mask of claim 1, wherein the pre-filter and the nanofiber layer comprise polyacrylonitrile nanofibers.

5. The PAP mask of claim 4, wherein the polyacrylonitrile nanofibers of the pre-filter and the nanofiber layer have a diameter of from 200 nm to 700 nm.

6. The PAP mask of claim 1, wherein the nanofiber layer having the anti-microbial catalyst incorporated therein has a thickness of from 0.01 mm to 0.5 mm.

7. The PAP mask of claim 1, further comprising an exhaust, wherein the exhaust includes a filter for filtering the air being exhaled by the wearer before the exhaled air reaches an atmospheric environment.

8. The PAP mask of claim 1, the main body of the PAP mask further comprising a divider that separates the PDC assembly, the fan assembly, and the pre-filter from electronic components.

9. The PAP mask of claim 1, wherein a volume flow rate of air through the PAP mask is at least 72 L/min.

10. The PAP mask of claim 1, further comprising a plasma generator, wherein the plasma generator provides a voltage of from 1 kV to 5 kV to the PDC assembly.
